(19) United States
(12) Patent Application Publication
(10) Pub. No.: US 2010/0317037 A1
(43) Pub. Date: Dec. 16, 2010

(54) Detection of Mycobacterium Tuberculosis Bacilli

(75) Inventor: Ahmad Bahrmand, Houston, TX (US)

Correspondence Address: D'AMBROSIO & MENON, LLP, 10260 WESTHEIMER, SUITE 465, HOUSTON, TX, 77042, US

(73) Assignee: Sina Biotechnical Co., Houston, TX (US)

(21) Appl. No.: 12/816,106

(22) Filed: Jun. 15, 2010

Related U.S. Application Data
(60) Provisional application No. 61/187,354, filed on Jun. 16, 2009.

(51) Int. Cl. G01N 33/569

(52) U.S. Cl. 435/7.32

(57) ABSTRACT

A screening test for Mycobacterium tuberculosis in patient samples includes an antibody preparation prepared against the cell wall antigens of M. tuberculosis. One embodiment of the screening test is rapid and has a negative predictive value of 95% or more. Furthermore, the use of an antibody preparation that will specifically bind to multidrug resistant strains of Mycobacterium tuberculosis bacilli permits the screening test to differentiate the detection of Mycobacterium tuberculosis bacilli strains that are resistant to the antibiotics rifampicin and isoniazid.

### CROSS-REFERENCE TO RELATED APPLICATION

The present application, pursuant to 35 U.S.C. 111 (b), claims the benefit of the earlier filing date of provisional application Ser. No. 61/187,354 filed Jun. 16, 2009, and entitled “Detection of Mycobacterium Tuberculosis Bacilli.”

### BACKGROUND OF THE INVENTION

1. Field of the Invention

The invention relates to a method for the detection of Mycobacterium tuberculosis in a patient sample. More particularly, the present invention relates to a method for the differential detection of the Mycobacterium tuberculosis bacilli strains that are resistant to the antibiotics rifampicin and isoniazid.

2. Description of the Related Art

Tuberculosis, or TB, is a common and often deadly infectious disease generally caused by Mycobacterium tuberculosis in humans. Infection with Mycobacterium tuberculosis is an important problem in the world and was declared a global health emergency in 1993 by the World Health Organization.

When discovered by Robert Koch in 1882, Mycobacterium tuberculosis was responsible for nearly one in seven deaths in Europe. Today, Mycobacterium tuberculosis remains one of the most common infectious diseases in adults throughout the world, and approximately eight million new tuberculosis cases are identified each year.

The nature of tuberculosis and the life cycle of the bacilli that causes TB render the diagnosis of mycobacterial infections hard to establish. General clinical signs are not really characteristic of tuberculosis and specific signs are rare and inconsistent, especially in the case of extra-pulmonary tuberculosis. Usually, the clinician suspects tuberculosis infection when they are faced with persistent symptoms that are not alleviated by standard treatment, or when they have dismissed all other possibilities.

When a case of tuberculosis (TB) is suspected, the physician generally performs a chest X-ray, an intra-dermal tuberculin skin test (TST), and an acid fast bacillus (AFB) microscopic examination and culture examination. Each of these test has its own set of problems.

The radiological picture of the lungs is variable and, although very suggestive, is not specific to tuberculosis. Furthermore, chest X-rays are limited to diagnosing pulmonary TB and is only useful in cases of long-standing active TB since lung sequellae are slow to develop.

The TST test also has its drawbacks. For example, it is positive in a large number of vaccinated individuals, it is non-specific and it is influenced by saprophytic environmental mycobacteria against which the subject may have generated an immune reaction. Finally, the TST test often gives false positive results in immune depressed individuals such as the elderly and HIV patients.

Thus, the current clinical and anatomic pathological signs are not specific enough to tuberculosis and only allow a presumptive diagnosis to be made. A definitive diagnosis must therefore be based on the detection of TB bacilli in pathological specimens. The World Health Organization (WHO) (1,2,3), the Centers for Disease Control and Prevention (CDC) (4) and the International Union Against Tuberculosis and Lung Diseases (IUATLD) (5, 6) have all published reports on tuberculosis testing. The reports discuss techniques in laboratory management, the different methods of decontaminating patient samples, and the culturing and microscopic examination of patient specimens accepted by those institutions. Over the last few decades, these three major institutions have collaborated to develop common TB diagnostic methodologies tailored to different parts of the World.

The development of a reliable diagnostic screening method for TB bacilli is faced with several inherent problems. First, tuberculosis-causing mycobacteria are very slow-growers and are difficult to isolate from samples contaminated by other microorganisms that overgrow the tubercle bacilli. Secondly, bacilli emission in sputum, the sample type used to detect pulmonary TB, is not continuous. Several samples (generally 3) must therefore be tested before tuberculosis infection can be excluded with reasonable confidence. Sputum is also difficult to obtain in certain groups of patients, such as children and the elderly.

Other problems are linked to the diagnostic methods themselves. For example, most patient samples to be tested are contaminated with other microorganisms. Because of the sheer number and rapid growth rate of the contaminating microorganisms they typically overgrow and mask the presence of the TB bacilli. The methods developed to clear the samples of non-TB microorganisms need to be aggressive, but such aggressive methods often eliminate a large part or the TB bacilli present in the decontaminated sample which often leads to false negative results.

The current gold standard for tuberculosis diagnosis is a bacterial culture on Lowenstein-Jensen (L-J) medium. However, this culture standard is not 100% sensitive, but rather has a 70% to 90% sensitivity in adult patients with known active pulmonary TB (7), and drops to between 18% and 50% in other patient groups and in cases of extra-pulmonary TB (8, 9). Another problem with a definitive tuberculosis diagnosis using culture techniques is the difficulty in culturing the slow-growing Mycobacterium tuberculosis in the laboratory. In fact, it may take 4 to 12 weeks to culture sufficient Mycobacterium tuberculosis from a sputum sample for a definitive TB diagnosis. Thus while culture remains the gold standard for TB detection, the length of time required before obtaining results and the equipment and media required to perform this technique makes it inadequate for use in most TB endemic areas.

To compensate for the length of time that cultures take, the direct observation of acid fast bacilli (AFB) under the microscope is often used. However, direct observation under the microscope is not specific for the tubercle bacilli, as all mycobacteria are acid fast. Microscopic examination of AFB is relatively sensitive in cases of active pulmonary TB with cavitation in adults (10) but it is reduced to 70% or even 25% when other TB types and other patient groups, such as young children, elderly people and HIV infected patients are considered (11, 12). On average, it is believed that 50% of the new cases of pulmonary TB are detected by AFB microscopic examination (13-14). For extra-pulmonary TB, the sensitivity of AFB microscopic examination depends on the infected organ and varies between 10-50% (15-16).

The World Health Organization (WHO) has promoted the diagnosis of TB in developing countries based on the microscopic identification of acid fast bacilli (AFB) in a sputum smear. AFB microscopy is inexpensive and, other than the microscope, requires little material and gives results in one day. On the other hand, it is a tiring and cumbersome technique that relies heavily on technicians' skill and human interpretation.

In an effort to reduce the sensitivity problems of the TB tests, the WHO has setup a validation and control system for all laboratories involved in TB diagnosis (2). Because the AFB test is non-specific and has a lower sensitivity, the direct AFB microscopic examination of samples cannot replace the culture test. Yet, the direct observation of results with the AFB test allows physicians to get a rapid indication that their early diagnosis is probably correct. This confirmation is often sufficient to initiate therapy while waiting for definite answers on the basis of culture.

The rise in infections and the neglect of TB control programs have enabled a resurgence of tuberculosis. In addition, multi-drug resistant tuberculosis (MDR-TB), a subset of TB that is resistant to two powerful anti-TB antibiotics isoniazid (INH) and rifampicin (RMP) has recently become a major global concern. MDR-TB typically develops during treatment of fully-sensitive TB when the course of antibiotics is interrupted and the levels of drug in the body are insufficient to kill 100% of bacteria. This can happen for a number of reasons: patients may feel better and halt their antibiotic course, drug supplies may run out or become scarce, or patients may forget to take their medication from time to time.

MDR-TB is more difficult and more expensive to treat than the normal antibiotic-sensitive TB. Thus, treatment of MDR-TB must be done on the basis of antibiotic-sensitivity testing in cultured bacilli.

Other more sensitive and specific diagnostic tools have been developed. For example, the identification of M. tuberculosis in a patient through the use of DNA probes and polymerase chain reaction (PCR) amplification has a sensitivity greater than 90% and a specificity close to 100% in smear positive cases, and is ideal for differentiating true TB infection from infection with non-tuberculosis mycobacteria. However, DNA probes are costly and not suitable for TB screening in countries with a high prevalence of TB.

Recently, immunoassays have been employed for the detection of one or more of the Mycobacterium tuberculosis antigens that are considered to be abundant extracellular antigens having a molecular weight of 110 kiloDaltons (KD), 80 KD, 71 KD, 58 KD, 45 KD, 32 KD, 30 KD, 24 KD, 23.5 KD, 23 KD, 16KD, 14 KD and 12 KD.

However, to date none of the immunoassays that are available to detect M. tuberculosis can differentiate whether the strain of M. tuberculosis that is infecting a patient is resistant or sensitive to antibiotic treatment. An accurate and rapid detection of whether a patient is infected with an antibiotic-sensitive strain of Mycobacterium tuberculosis or an antibiotic resistant strain of M. tuberculosis is an important step in combating tuberculosis around the world.

Currently, the laboratory diagnosis of MDR-TB is typically based on antibiotic sensitivity testing. Although a gene probe for rpoB (rifampicin sensitivity) is available in some countries, the requisite gene amplification technology requires a sophisticated laboratory and is not suitable for most regions having a high incidence of MDR-TB.

Accordingly, there is an ongoing need for a quick, sensitive, and less expensive detection of M. tuberculosis in a clinical sample.

### SUMMARY OF THE INVENTION

The invention relates to a screening test and its method of use for the detection of Mycobacterium tuberculosis in a patient sample, wherein the screening test contains an antiserum prepared against the cell wall antigens of M. tuberculosis.

One embodiment of the invention is a rapid screening test for M. tuberculosis that has a negative predictive value of 95% or more.

Another embodiment of the screening test uses an antiserum preparation that will specifically bind to multidrug resistant strains of Mycobacterium tuberculosis bacilli, wherein the screening test can differentiate the detection of Mycobacterium tuberculosis bacilli strains that are resistant to the antibiotics rifampicin and isoniazid.

Yet another embodiment of the invention is an immunoassay for screening patient samples for Mycobacterium tuberculosis, the immunoassay having an antiserum against a plurality of cell wall antigens isolated from a multitude of Mycobacterium tuberculosis strains.

Still another embodiment of the invention is an immunoassay for screening patient samples for a multidrug resistant strain of Mycobacterium tuberculosis, the immunoassay having an antiserum against a plurality of cell wall antigens isolated from a multitude of multidrug resistant Mycobacterium tuberculosis strains.

The foregoing has outlined rather broadly several embodiments of the present invention in order that the detailed description of the invention that follows may be better understood. Additional features and advantages of the invention will be described hereinafter which form the subject of the claims of the invention. It should be appreciated by those skilled in the art that the conception and the specific embodiment disclosed might be readily utilized as a basis for modifying or redesigning the structures for carrying out the same purposes as the invention. It should be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 is a flow diagram showing the basic steps in isolating the cell wall antigens from M. tuberculosis cells.

FIG. 2 is a flow diagram showing the basic steps in the purification of the cell wall antigens.

FIG. 3 is a flow diagram showing the basic steps in the preparation of the plate for absorbing unwanted antibodies from the antiserum against the M. tuberculosis cell wall antigens.

FIG. 4 is a flow diagram showing the basic steps in the purification of the antiserum prepared against the cell wall antigens to the antibiotic sensitive strains of M. tuberculosis.

FIG. 5 is a flow diagram showing the basic steps in the purification of the antiserum prepared against the cell wall antigens to the multidrug resistant strains of M. tuberculosis.

FIG. 6 is a flow diagram showing the basic steps in the TB Rapid Test.

FIG. 7 is a photograph of the TB Rapid Test results for known positive and negative samples using the antiserum made against the antibiotic sensitive strains of M. tuberculosis.

FIG. 8 is a photograph of the TB Rapid Test results for known positive and negative samples using the antiserum made against the multidrug resistant strains of M. tuberculosis.

Pursuant to §1.84(a)(2), this application file contains at least one drawing executed in color. Copies of this patent application publication with color drawings will be provided by the Office upon request and payment of the necessary fee.

### DETAILED DESCRIPTION

The present invention relates to a method for the detection of Mycobacterium tuberculosis, or M. tuberculosis, in a patient sample. More particularly, the present invention relates to a method for the differential detection of the Mycobacterium tuberculosis bacilli strains that are resistant to antibiotics and the bacilli strains that are sensitive to antibiotics.

A. Establishment of Specimen Banks

A M. tuberculosis negative sputum bank was generated (direct microscopy negative, culture negative, PCR negative). Numerous (about 300) human sputum samples were randomly collected from individuals in different geographical locations to ensure a wide variety of human mouth microflora. The sputum samples were decontaminated by the KUBICA method. This decontamination method is accepted by the CDC, IUATLD and WHO. It is the most common method used in mycobacterial labs and involves the use of a 4% sodium hydroxide (NaOH) solution mixed with a 2.9% citrate solution and N-acetyl-L-cysteine as a liquefying agent. This method provides good decontamination of the sample without over-killing the TB bacilli in the sample. The decontaminated sputum samples were tested for M. tuberculosis by AFB smear microscopy, antibiotic sensitivity culture techniques, and PCR methods. The TB negative sputum samples were used as a negative sputum bank (NSB).

The decontaminated sputum samples were also used to culture numerous M. tuberculosis strains. The M. tuberculosis bacilli strains were collected from a wide variety of geographic areas known to have either a high incidence or a low incidence of tuberculosis. The various M. tuberculosis strains were isolated, cultured and tested for sensitivity to anti-mycobacterium drugs using standard Center of Disease Control (CDC) sensitivity testing methods.

An antibiotic-sensitive M. tuberculosis bacilli (ASMTB) bank was generated by collecting at least 100 tubercle bacilli strains proven to be sensitive to anti-mycobacterium drugs using standard anti-mycobacterium drug sensitivity tests.

Similarly, a multidrug resistant M. tuberculosis bacilli (MDRTB) bank was generated by collecting at least 100 tubercle bacilli strains proven to be resistant to the anti-mycobacterium drugs rifampicin and isoniazide using standard CDC drug sensitivity testing methods. Single isolated MDR-TB strains were grown on LJ medium and their cell wall antigens were selectively induced by passing the MDR-TB strain multiple times on LJ media having increasing concentrations of rifampicin and isoniazid. Typically, the resistance of the MDR-TB isolated strains was increased by growing the isolates on LJ media with rifampicin/isoniazid concentrations that were incrementally increased with each passage from 80 μg/ml rifampicin and 2 μg/ml isoniazid to 160, 320 or 400 μg/ml rifampicin and 4, 8, or 16 μg/ml isoniazid.

The induced cell wall antigens of the selectively multidrug resistant M. tuberculosis bacilli generally comprised one or more proteins having a molecular weight of 18.5 KD, 44 KD, 66.2 KD, 76 KD, 116 KD, or 135 KD. For example, in one preparation of the cell wall antigens of the MDR-TB strains, the 18.5 KD and the 44 KD protein concentrations were increased about four fold by the enhancement process and the concentration of the 66.2 KD, 76 KD, 116 KD, and 135 KD proteins were generally increased from about two to four times the concentration found in MDR M. tuberculosis bacilli strains that were not induced with rifampicin and isoniazid.

The antibiotic-sensitive M. tuberculosis bacilli (ASMTB) hank and the multidrug resistant M. tuberculosis bacilli (MIDRIB) bank were used to isolate and purify diverse antigens specific for the antibiotic sensitive M. tuberculosis bacilli and for the multi-drug resistant M. tuberculosis bacilli. The specific antigens purified from the antibiotic sensitive and the MDR tubercle bacilli were then used to generate and purify antibodies specific for either the antibiotic sensitive M. tuberculosis bacilli, or for multidrug resistant M. tuberculosis bacilli.

A unique and sensitive immunoassay was developed using the specific antigenic materials and the specific antibodies generated from those material. The immunoassay described herein can quickly identify not only the presence of the M. tuberculosis bacilli in a patient specimen, but can also identify whether or not the patient specimen contains a multidrug resistant M. tuberculosis bacilli strain.

B. Isolation and Purification of Cell Wall Antigens

Cell wall antigens having a molecular weight of 18.5 kiloDaltons (KD), 44 KD, 66.2 KD, 76 KD, 116 KD, and 135 KD were isolated from lysed tubercle bacilli using gel filtration and visualized using SDS-PAGE gel electrophoresis. Cell wall antigens were isolated and purified from both the antibiotic sensitive specimen bank (ASMTB) and from the MDR specimen bank (MDRTB).

M. tuberculosis has an unusual, waxy coating on the cell surface primarily composed of the lipid mycolic acid. The unusual cell wall is likely responsible for making the cells impervious to Gram staining so that acid-fast detection techniques are used instead. Furthermore, the high concentration of lipids in the cell wall of M. tuberculosis has contributed to the previous inability to obtain a consistently high quality preparation of cell wall antigens.

A novel process has been developed to remove and purify a high quality, high titer antigenic preparation from M. tuberculosis bacilli. Surprisingly the method has provided the isolation of a significant quantity of tubercle protein that is not denatured by the process of isolation.

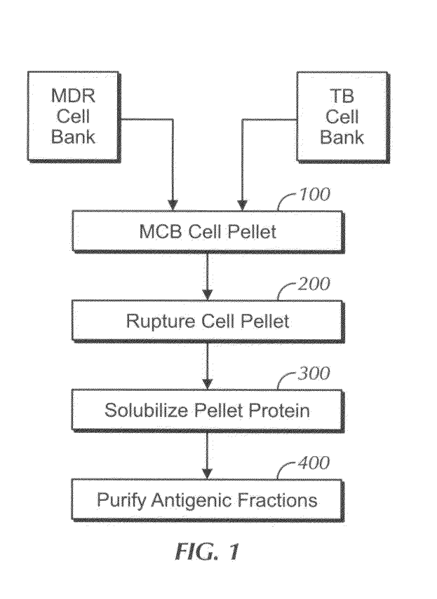

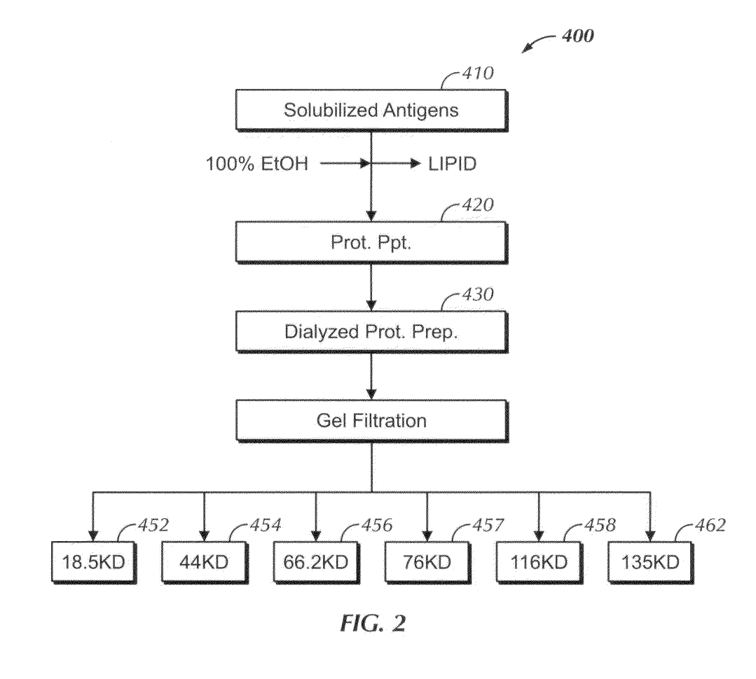

FIG. 1 is a flow chart of isolation process and FIG. 2 is a flow chart of the protein purification procedure. The starting material for the M. tuberculosis antigenic preparation was a washed M. tuberculosis cell pellet derived from a cell bank composed of about 100 different strains of tubercle bacilli.

The tubercle cell pellet, step 100 of FIG. 1, is processed from numerous tubercle bacilli colonies that were harvested and transferred to sterile 50 ml conical test tubes. The tubercle bacilli underwent numerous washings (generally at least 4 washings) with double distilled water. Typically, the tubercle bacilli were washed in 50 ml conical tubes by adding 50 ml of double distilled water to the bacilli, suspending the bacilli in the water, centrifuging the suspension at 4000 RPM for 30 min, and then discarding the supernatant.

The tubercle bacilli and its cell wall were then ruptured, step 200 of FIG. 1, by subjecting the cell pellet to repeated dramatic temperature changes. Generally, the washed tubercle bacilli pellet was suspended in 5 ml of double distilled water, frozen in liquid nitrogen for 5 to 10 minutes, and then immediately transferred to 100° C. boiling water. The dramatic alternating cold/hot temperature changes were repeated about ten times in order to rupture the tubercle bacilli and its cell wall.

The antigens were solubilized from the ruptured tubercle bacilli in step 300. The solubilisation of the M. tuberculosis antigens was accomplished by sonication of the ruptured tubercle bacilli in the presence of a buffered detergent. Basically, the ruptured cells were suspended in 5 ml of buffer and mixed with 5 ml of sonication buffer containing detergent. The sonication buffer typically contained 100 ml of phosphate buffered saline (PBS), 0.02 g/l of sodium azide, 10 ml of a 1% v/v solution of Triton X-100, and 10 ml of glycerol containing 10 mM dithiothreitol (DTT) 1 mM phenylmethane sulfonyl fluoride (PMSF), and 20 mM ethylenediamine tetraacetic acid (EDTA).

Approximately equal volumes of the ruptured tubercle bacilli suspension and sonication buffer were sonicated for about 10 minutes at about 50 to 60 milliamp (mA) power per cycle. The non-solubilized cell debris was then removed by centrifugation at 12,000 rpm at 4° C.

Solubilized M. tuberculosis antigenic fractions were then purified (step 400) as illustrated in FIG. 2. First the solubilized proteins 410 were concentrated by precipitation with cold (−20° C.) absolute 100% ethanol (EtOH). The protein precipitate 420 was prepared by placing the solubilized protein fraction from step 300 in a −20° C. freezer over an ice container and slowly adding cold absolute EtOH drop by drop up to 60% of the total volume. The EtOH containing mixture was incubated at −20° C. for about 1 hour to encourage the precipitation of protein. The entire mixture was then centrifuged at −20° C. for 30 minutes and the lipid containing supernatant discarded.

The protein precipitate 420 was placed in dialysis tubing having about a 3000 molecular weight cutoff. The protein containing dialysis tubing was then dialyzed for about 3 hours against about 2 liters of 10 mM phosphate buffer, containing 0.15 M sodium chloride (NaCl), at pH 7.6 in order to remove the alcohol. The dialyzed protein preparation 430 was stored at −80° C. The protein concentration of the dialyzed protein preparation 430 was calculated according to a modified Bradford protein assay.

The dialyzed protein preparation 430 was then chromatographed on a 120 ml column (60 cm×1.6 cm) of Sephadex G-100 or Sephadex G-75 to separate different molecular weight fractions. The dialyzed protein preparation 430 was applied to the column in approximately a 6 ml sample having about 58 mg of protein/ml of 50 mM Tris-HCl buffer, pH 6.8, containing 0.5 M NaCl. The void volume of the column was about 43-45 ml and the flow rate of the buffer was about 1.0-1.5 ml/min. The eluate was collected in 2 ml samples and the protein concentration of each sample tested using the modified Bradford protein assay. The protein molecular weight of the eluted fractions was calculated using blue dextran and phenol red markers.

Six fractions of protein containing eluate were collected. These fractions contained proteins having molecular weights of 18.5 KD (452), 44 KD (454), 66.2 KD (456), 76 KD (457), 116 KD (458), and 135 KD (450). Each protein fraction was dialyzed against a 10 mM phosphate buffer, containing 0.15 M sodium chloride (NaCl), at pH 7.6. The dialyzed protein fractions were stored at −80° C. until use.

The protein concentration of the 18.5 KD (452), 44 KD (454), 66.2 KD (456), 76 KD (457), 116 KD (458), and 135 KD (450) protein fractions was calculated and the homogeneity of the protein in each fraction was visualized using sodium dodecyl sulphate polyacrylamide gel electrophoresis (SDS-PAGE) on a 12.5% agarose gel.

C. Antibody Production and Initial Purification

Antibody production was induced in a mammal by injecting the purified M. tuberculosis protein fractions isolated from the antibiotic sensitive M. tuberculosis cultures and the multi-drug resistant M. tuberculosis cultures. The purified protein antigens 18.5 KD, 44 KD, 66.2 KD, 76 KD, 116 KD, and 135 KD from the antibiotic sensitive cultures were typically mixed to induce antibodies specific to various antigenic determinants that were specific for the antibiotic sensitive tubercle bacilli strains. Similarly, the purified protein antigens 18.5 KD, 44 KD, 66.2 KD, 76 KD, 116 KD, and 135 KD from the MDR-TB cultures were typically mixed to induce antibodies specific to various antigenic determinants that were specific for the MDR tubercle bacilli strains.

Although various protocols are used for antibody induction in mammals, one protocol used included the induction of antibodies in twelve weeks old male rabbits using the injection schedule set forth below.

Briefly, 250 μg of the purified M. tuberculosis antigens were mixed with 250 μg of Freund's incomplete adjuvant and injected subcutaneously in a male rabbit on day 1 of the injection schedule. On day 21, a second subcutaneous injection having 204 μg of the M. tuberculosis antigen preparation was mixed with 100 μg PBS and 250 μg of Freund's incomplete adjuvant was injected subcutaneously into the same male rabbit. On day 42, a third subcutaneous injection having 175 μg of the M. tuberculosis antigen preparation was mixed with 175 μg PBS and 300 μg of Freund's incomplete adjuvant was injected subcutaneously into the same male rabbit. Finally on day 64, a fourth subcutaneous injection having 175 μg of the M. tuberculosis antigen preparation was mixed with 175 μg PBS and 300 μg of Freund's incomplete adjuvant was injected subcutaneously into the same male rabbit.

On day 67, the rabbit was bled via a heart puncture to obtain 10-25 ml of blood. The rabbit blood was clotted and the serum separated and stored at 4° C. The serum was then titrated to determine its reactivity with the injected antigen preparation.

The antibody fraction of the rabbit serum was initially purified by affinity chromatography. There are several proteins that are known to bind to antibodies and have been used to separate the immunoglobulin fraction of serum from other proteins. One embodiment of the present invention uses an affinity column having Protein G immobilized on Sepharose 4B

Thus, immunoglobulin fractions that are highly specific for either the antibiotic sensitive strains of M. tuberculosis bacilli or the MDR resistant strains of M. tuberculosis bacilli are generated.

D. Enhancement of Antibody Specificity

Highly specific antibodies were produced using a unique absorption process for removing many unwanted antibodies within the immunoglobulin fractions that exhibit unwanted binding to non-selected antigenic determinants. By eliminating these unwanted non-specific antibodies, highly specific antibodies are generated that are used in a fast assay for M. tuberculosis bacilli.

1. Antibodies Specific for M. tuberculosis Bacilli

The generation of a first specific antibody preparation that will bind with the cell wall proteins of both the antibiotic sensitive strains of M. tuberculosis bacilli and the MDR resistant strains of M. tuberculosis bacilli is described (Ab-AS/MDR). The Ab-AS/MDR preparation 650 is used in an immunologically based assay to indicate the presence of M. tuberculosis bacilli in patient samples.

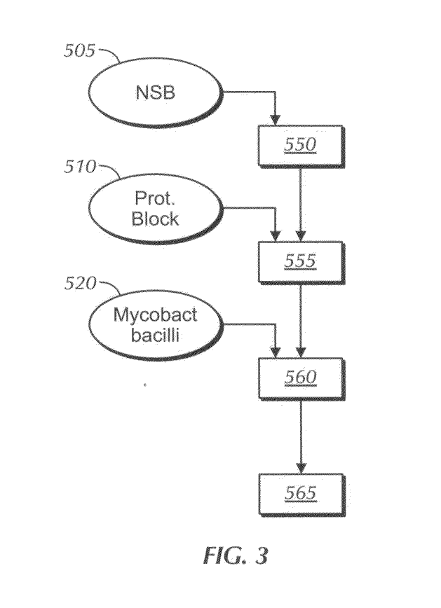

Protein blocked 96 well Elisa plates 560 were prepared as illustrated in FIG. 3. A sample of the decontaminated negative sputum hank (NSB) 505 described above was adjusted to a pH of 9.0 with sodium bicarbonate. A multichannel pipette was used to pipette 150 μl of the NSB 505 adjusted to pH 9.0 into each well of a new 96 well ELISA plate 550. The plate and the NSB 505 were incubated at 4° C. for about 48 hours. A portion of the protein in the decontaminated NSB 505 was absorbed to the plate 550 to form a plate 555 having a first absorbed protein. The unabsorbed unwanted excess supernatant was removed by flipping the plate 555 over onto a dry piece of absorbent paper.

Next 110 μl of a blocking protein solution 510 was added to each well of the plate 555. The blocking protein solution 510 contained 1 gram of bovine albumin fraction V and 0.05 gm of sodium azide in 100 ml of distilled water. The blocking protein solution 510 was incubated in the wells of the plate 555 for 48 to 72 hours at 4° C. to form a plate 560 having a protein blocked surface. The unabsorbed unwanted excess supernatant was removed by flipping the plate 560 over onto a dry piece of absorbent paper.

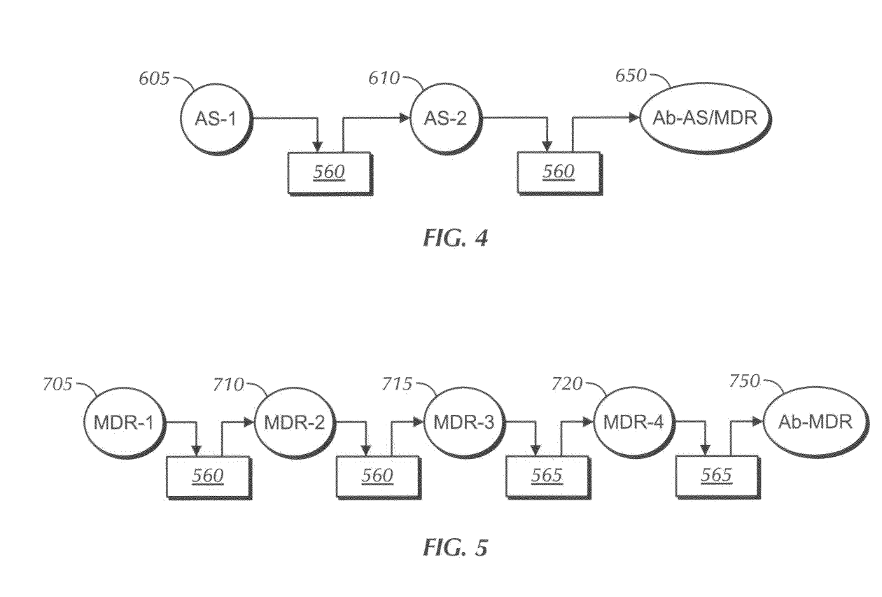

Protein blocked plates 560 were used to generate a first specific antibody preparation that will bind with the cell wall proteins of both the antibiotic sensitive strains of M. tuberculosis bacilli and the MDR resistant strains of M. tuberculosis bacilli (see FIG. 4). A first step was to add 100 μl of the immunoglobulin fraction (AS-1) 605 prepared using cell wall protein extracted from the antibiotic-sensitive strains of M. tuberculosis bacilli (ASMTB) to each of the 96 wells of the blocked plate 560 and incubate the plate at 25-30° C. for 3 hours while being swirled very slowly clockwise and counterclockwise ten times every 15 min.

The non-adhered antibodies (AS-2) 610 in the supernatant were carefully removed using a multichannel pipette and stored in a sterile glass container. Generally about 30%-35% of the antibodies in AS-1 will adhere to the plate 560 and about 65%-70% of the antibodies are removed in AS-2.

The adherence procedure described above is repeated using the AS-2 non-adhered antibodies 610 that were removed from the first plate 560. Basically the AS-2 preparation 610 was incubated with a second plate 560 and approximately 10% of the antibodies in the AS-2 were adhered to the plate 560 during the second absorption. Thus, the final Ab-AS/MDR antibody reagent 650 only had about 55%-60% of the antibodies that were present in the AS-1. The final Ab-AS/MDR reagent 650 specifically binds to both the cell wall antigens of the antibiotic sensitive M. tuberculosis bacilli and the MDR M. tuberculosis bacilli.

2. Antibodies Specific for MDR M. tuberculosis Bacilli

Antibodies that are specific for MDR M. tuberculosis bacilli (Ab-MDR) were prepared as described below. The initial steps in the process are similar to the process used in the production of the Ab-AS/MDR. The Ab-MDR preparation 750 is used in an immunologically based assay to indicate the presence of MDR strains of M. tuberculosis bacilli in patient samples.

Protein blocked plates 560 were prepared as shown in FIG. 3 and were used in the initial steps in the production of the Ab-MDR 750 (see FIG. 5). A first step was to add 110 μl of the immunoglobulin fraction (MDR-1) 705 prepared using cell wall protein extracted from the multidrug resistant strains M. tuberculosis bacilli (MDRTB) to each of the 96 wells of a first blocked plate 560 and incubate the plate at 25-30° C. for 3 hours while swirling the plate very slowly clockwise and counterclockwise ten times every 15 min.

The non-adhered antibodies (MDR-2) 710 in the supernatant were carefully removed using a multichannel pipette and stored in a sterile glass container. Generally about 30%-35% of the antibodies in MDR-1 will adhere to the first plate 560 and about 65%-70% of the antibodies are removed in MDR-2.

The adherence procedure described above is repeated with the MDR-2 non-adhered antibodies that were removed from the first plate. Basically the MDR-2 preparation 710 was incubated with a second plate 560 and approximately 10% of the less specific MDR antibodies were absorbed on the second plate 560, leaving approximately 55%-60% of the antibodies from MDR-1 in the collected supernatant MDR-3 715.

Then 5×108 colony forming units (CFU)/ml of the ASMTB bank were mixed with 5×108 CFU/ml of Bacillus Calmette-Guerin (BCG) cells (a strain of Mycobacterium bovis) in one liter of 0.2 M sodium bicarbonate solution, pH 9.0. An 110 μl sample of this concentrated mixture of mycobacterium was added to each of the 96 wells of protein blocked plates 560 and incubated at 37° C. for 2 hours to produce plates 565 having mycobacterium antigens absorbed on the plates 565. The plates 565 were stored at 4° C. The excess liquid was removed by flipping over the plate onto a dry piece of absorbent paper.

An 110 μl sample of the MDR-3 antibody preparation 715 was added to each of the 96 wells of a first plate 565 and incubated at 25-30° C. for 3 hours while swirling the plate very slowly clockwise and counterclockwise ten times every 15 min. The non-adhered antibodies (MDR-4) 720 in the supernatant were carefully removed using a multichannel pipette and stored in a sterile glass container. Generally an additional 30%-35% of the antibodies in the MDR-3 preparation will adhere to the plate 565.

Finally, the adherence procedure described above for the MDR-3 antibody preparation is repeated with the MDR-4 non-adhered antibodies 720 that were removed from the first plate 565. Approximately 10% of the antibodies in the MDR-4 preparation are adhered to the second plate 565 leaving only about 20% of the antibodies of the original MDR-1 705 antibodies in the final Ab-MDR antibody reagent 750. The final Ab-MDR reagent 750 binds very specifically to MDR M. tuberculosis bacilli and not to the cell wall antigens of the antibiotic sensitive M. tuberculosis bacilli.

E. TB Screening Assay

The Ab-AS/MDR antibody reagent 650 and the Ab-MDR antibody reagent 750 were used to develop a quick screening assay for TB. The TB Rapid Test allows for the direct observation of test results. The TB Rapid Test detects mycobacterium bacilli retained on a filter using specific antibodies directed against the M. tuberculosis. The presence of these specific antibodies is visualized using an immunoglobulin binding reporter conjugate. A preferred embodiment of the reporter molecule is gold micellae that can be directly visualized. The binding of the gold micellae conjugate to antigenic determinants retained on a filter is evidenced by the appearance of a red-pink dot on the filter indicating the presence of M. tuberculosis bacilli in the patient specimen.

The TB Rapid Test does not require the typical 15 minutes of slide observation required to validate the negativity of a sample using the AFB microscopy technique. In contrast to the AFB microscopy or the culture methods, the results of the TB Rapid Test are read in a few seconds.

The TB Rapid Test method uses an approved decontamination method for patient sample. Once decontaminated, the patient sample can be used to start cultures, make microscopic smears, and perform the TB Rapid test. The TB Rapid test can also be used with samples other than expectorations, such as CSF, pleural fluid, pus, lymph node aspirates, biopsies, and the test can even be used directly with cultures to confirm the mycobacterial identity of the isolated pathogen.

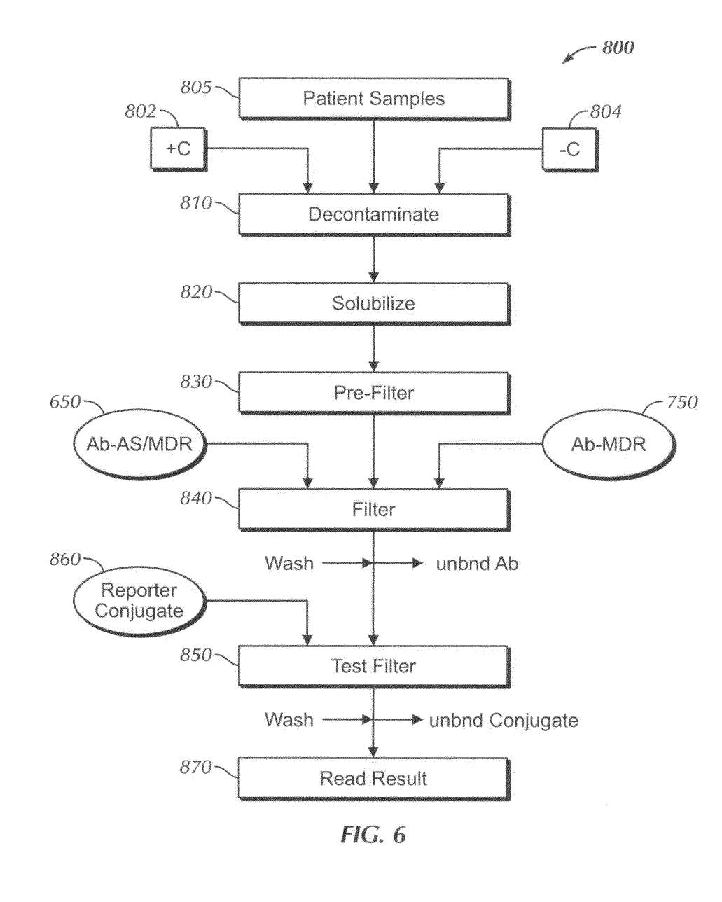

The TB Rapid Test method, shown schematically in FIG. 6, basically includes the steps of: (1) the decontamination of the patient samples; (2) further dissolving the test samples so that the samples can pass through a pre-filter; (3) passing the dissolved test sample through a pre-filter to remove any large particles and allowing the pre-filtered dissolved test sample to absorb on a test filter; (4) adding specific M. tuberculosis bacilli binding antibodies (either Ab-AS/MDR 650 or Ab-MDR 750) to the test filter and allowing the antibodies to bind if the appropriate M. tuberculosis bacilli are present; (5) adding a reporter conjugate to visualize bound antibody. The TB Rapid Test is positive for the presence of the appropriate M. tuberculosis bacilli if a red-pink dot appears in the middle of the test filter.

As shown in step 810 of FIG. 6, all positive controls, all negative controls, and all patient samples must be decontaminated. The preferred embodiment of the decontamination method is the Kubica method, which is the most widely used decontaminating methodology and is accepted by the CDC, IUATLD and WHO. This decontamination method involves using a decontamination solution containing a 4% sodium hydroxide (NaOH) solution mixed with a 2.9% citrate solution and N-acetyl-L-cysteine as a liquefying agent.

The sample to be decontaminated is well mixed with the decontamination solution, incubated for 20 minutes on an agitator, then neutralized and centrifuged to separate the particulate pellet and the supernatant. The supernatant is discarded and the particulate pellet (including the tubercle bacilli) is used for AFB microscopy, PCR analysis, culture techniques, and the TB Rapid Test.

Preferred embodiments of the positive controls 802 consist of 3×107 live TB bacilli suspended in 0.1 ml of either negative sputum (NSB 505) or PBS. If the TB Rapid Test is being performed as a screening test for TB then the positive control may be from the ASMTB bank or mycobacterium bacilli preparation 520. However, if the TB Rapid Test is being performed to determine if the TB is multidrug resistant or not, then a preferred embodiment of the positive control is MDRTB bank.

Negative controls 804 must have no detectable M. tuberculosis bacilli in the sample. Preferred embodiments of negative controls include the NSB 505, PBS, or patient sputa that was shown to be TB negative by AFB microscopy. PCR analysis and culture techniques.

Patient samples 805 are typically sputa, broncho-alveolar aspirates, gastric washes and other sample types containing microbiological cells and must be decontaminated before used for tuberculosis diagnosis. One advantage of the TB Rapid Test is that it assesses patient samples that have been decontaminated using the same techniques required for any TB diagnostic procedure.

The particulate pellet from the decontaminated samples must be further solubilized in step 820 to allow the sample to pass through a glass fiber pre-filter to remove any large particulate matter that would clog the filter cartridge. Some patient samples are very viscous or have a number of particles in them, so all of the decontaminated samples are re-suspended, vortexed with a fresh dissolving solution, and boiled before passing it through the pre-filter.

The decontaminated, dissolved patient sample (generally 100 μl to 200 μl) is pre-filtered 830 by adding the sample to the center of a glass pre-filter that has been placed on top of a filter cartridge. Once the sample has passed through the pre-filter and into the filter cartridge 840, the pre-filter is washed with a small amount of washing fluid and then carefully removed from the filter cartridge.

One embodiment of a filter cartridge includes a 0.45 nm nitrocellulose membrane laid over an absorbing pad. The decontaminated, dissolved patient sample typically takes 30 seconds to 5 minutes to absorb into the filter cartridge. Once the sample is fully absorbed into the filter cartridge 840, a small amount of the specific antibody solution is added to the filter to bind to the M. tuberculosis antigens present on the filter cartridge.

If the TB Rapid Test is being used as a TB screening test then the Ab-AS/MDR 650 preparation is used. The Ab-As/MDR 650 will bind to both the antibiotic sensitive and the multidrug resistant tubercle bacilli antigens, which makes it a good screening method for TB. However, if the TB Rapid Test is being used to indicate whether or not a patient has a MDR stain of TB then the Ab-MDR 750 preparation is used. The Ab-MDR 750 will generally only hind to the MDR tubercle bacilli antigens.

The antibody reacted test filter 850 is washed with a small amount of washing solution and then a reporter conjugate solution 860 is added to the washed test filter 850. Unbound reporter conjugate 860 is rinsed off the test filter 850, leaving only reporter conjugate that is specifically bound to antibodies that have been specifically bound to the test sample.

Many different reporter molecules are usable in the TB Rapid Test. Typically the reporter molecule selected will be selected from the group comprising enzymes, chromophores, stained latex beads, fluorophores, gold micellae, and the like. Although any reporter conjugate can be used, a preferred embodiment of the reporter conjugate is a Protein A labeled gold micellae conjugate purchased from British Biocell (Cardiff, UK). The Protein A will specifically bind to immunoglobulins and the gold micellae conjugate is visually observable as a red/pink color. Other reporter conjugates are more sensitive; however, many of the more sensitive reporter type molecules require extra equipment (such as a spectrometer) to read the results and are not necessarily usable in the field.

The results should be read, step 870, and recorded immediately after the completion of the TB Rapid Test. When the gold micellae conjugate is used as the reporter conjugate a red/pink color at the reacted filter center indicates the presence of tubercle bacilli in the processed sample. It is recommended that the results of the TB Rapid Test be immediately recorded as the red/pink color may fade upon drying. A white color at the reacted filter center indicates a negative, or a lack of the tubercle bacilli in the processed sample. A negative test result may also show a light pink ring around the external circumference of the reacted filter, but it will have a whiter filter center.

One embodiment of the TB Rapid Test kit includes (1) a positive control, (2) a negative control, (3) a specific antibody preparation for binding M. tuberculosis antigens, and (4) a reporter conjugate.

Another embodiment of the TB Rapid Test is provided as a kit containing the following components.

1. A Filtration System
54 filtering cartridges
54 funnels
60 glass-fiber pre-filters

2. A Decontaminating System
1 vial of 4% NaOH Solution (64 ml)
1 vial of 2.9% Citrate Solution (64 ml)
1 vial of 20 fold concentrate Neutralizing Solution (60 ml)
2 tubes of 23% N-acetyl-L-cysteine solution (1.5 ml each)

3. The Rapid Test Components
3 tubes of Dissolving Solution (2.2 ml each)
2 dropper vials of Wash Solution (6.5 ml each) preserved with sodium mercurothiolate
2 dropper vials of Antibody Solution (3.75 ml each) preserved with sodium mercurothiolate
2 dropper vials of Gold Conjugate Solution (3.75 ml each) preserved with methylisothiazole and bromonitrodioxane.
2 dropper vials of Rinse Solution (6.5 ml each) preserved with sodium mercurothiolate
3 tubes of freeze-dried Positive Control preserved with sodium azide
1 tube of Negative Control (1 ml) preserved with sodium azide

The TB Rapid Test described above was evaluated by comparing the results of the TB Rapid Test obtained from patient sputum samples of patients suspected of having TB, with the results obtained on the same sputum samples by AFB microscopy, culture methods, and PCR analysis.

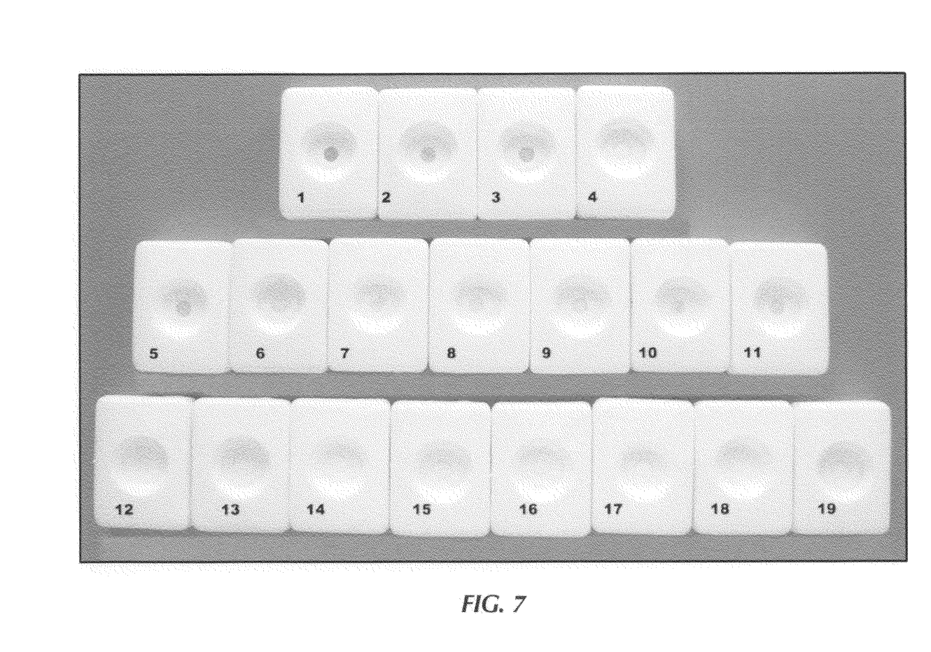

FIG. 7 shows the TB Rapid Test results when the Ab-AS/MDR 650 preparation is used. The Ab-As/MDR 650 will bind to both the antibiotic sensitive and the multidrug resistant tubercle bacilli antigens, which makes it a good screening method for TB.

The samples tested on the filter cartridges 1-19 shown in FIG. 7 are positive controls (samples 1-3), a negative control (sample 4), six patient sputa determined to be positive by AFB microscopy (samples 5-10), a negative sputa spiked with 50,000 tubercle bacilli (sample 11), seven patient sputa determined to be negative by AFB microscopy (samples 12-18), and the negative sputa used in sample 11 but unspiked (samples 19). The negative sputa appear white (samples 12-19) indicating no tubercle bacilli in the samples, whereas the positive controls (samples 1-3 and 11) and the positive sputa samples (samples 5-10) show a central red disk indicating the presence of tubercle bacilli. AFB microscopy and culture methods confirmed that the samples 5-10 were TB positive.

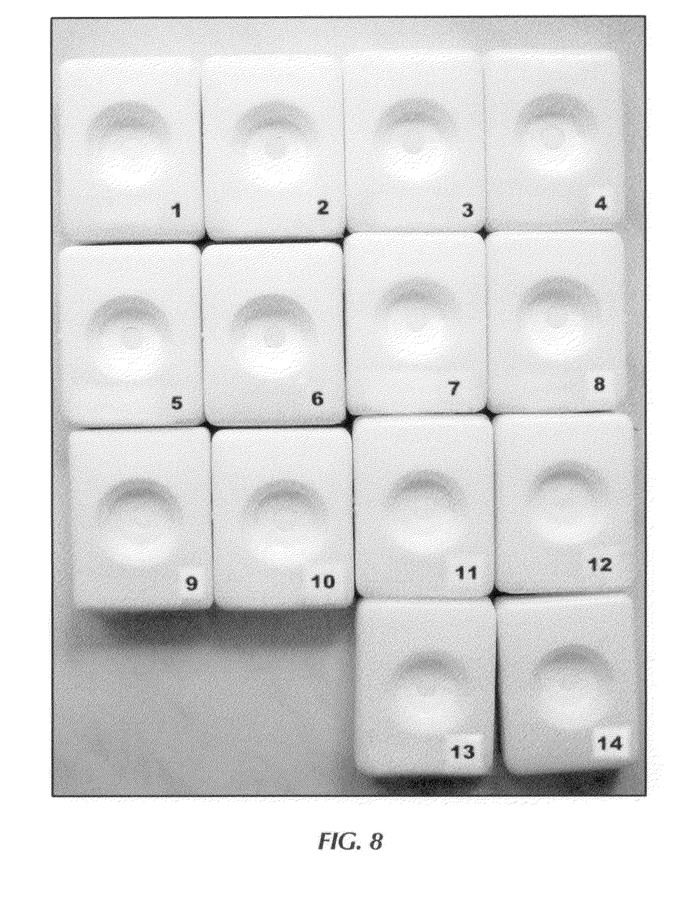

FIG. 8 shows the TB Rapid Test results when the Ab-MDR 750 preparation is used. The Ab-MDR 750 will generally only bind to the MDR tubercle bacilli antigens. The initial test results shown in FIG. 8 indicate that the TB Rapid Test using the Ab-MDR preparation 750 provides a good indication whether or not a patient has a MDR stain of TB.

The samples tested on the filter cartridges 1-14 shown in FIG. 8 are a BCG negative control (sample 1), positive controls (samples 2 and 5), four patient sputa determined to have MDR M. tuberculosis strains by culture methods and PCR analysis (samples 3-4, 7, and 13), and five patient sputa determined to be antibiotic sensitive by culture methods and PCR analysis (samples 9-12 and 14). Two patient sputa (samples 6 and 8) tested positive for MDR bacilli in the TB Rapid Test but actually contained TB that was resistant to rifampicin and sensitive to isoniazid. The negative sputa appear to have a white center (samples 9-12 and 14) indicating the tubercle bacilli are not MDR, whereas the MDR positive sputa samples (samples 3-4, 6-8, and 13) show a central red disk indicating the presence of MDR tubercle bacilli.

The definition of MDR tubercle bacilli is bacilli that are resistant to both rifampicin and isoniazid. The negative control (sample 1) had BCG cells or Mycobacterium bovis cells and was a true negative. Samples 9-12 and 14 were streptomycin resistant and sample 11 was kanamycin resistant, but none of these samples were resistant to rifampicin or isoniazid. Two patient sputa (samples 6 and 8) tested positive for MDR bacilli by the TB Rapid Test but actually contained TB that was resistant to rifampicin and sensitive to isoniazid and therefore did not fall within the definition of MDR.

F. Results of Clinical Trials

Clinical trial 1: A first clinical trial was performed in the Afghanistan-Iran border area where a large number of suspected TB cases are found. The clinical trial 1 was performed on 38 patient samples from suspected cases of TB. The diagnosis of TB in this clinical trial was established with clinical symptoms and a PCR positive test. The patient samples were evaluated with both the TB Rapid Test using Ab-AS/MDR and with an AFB microscopic evaluation performed the same day. The results of this clinical trial are presented in Table 1 below.

TABLE 1
A Comparison of the Diagnostic Value of the TB Rapid Test and
AFB Microscopy

| | True | False | True | False | | | | | |
|---|---|---|---|---|---|---|---|---|---|
| Diagnostic test | pos. | pos. | neg. | neg. | Sensitivity | Specificity | PPV* | NPV* | Efficiency |
| DIAGNOSIS | 27 | 0 | 11 | 0 | — | — | — | — | — |
| AFB stain | 8 | 2 | 9 | 19 | 29.6 | 81.8 | 80.0 | 32.1 | 44.7 |
| New TB rapid test | 17 | 3 | 8 | 10 | 63.0 | 72.7 | 85.0 | 44.4 | 65.8 |

*PPV = Positive Predictive Value, NPV = Negative Predictive Value

The AFB microscopy and the TB Rapid Test exhibited about the same rate of false positives 2 cases versus 3 cases respectively. However, the false negative rate from AFB microscopy (19 cases) was almost double the false negative rate of the TB Rapid Test (10 cases).

Clinical trial 2: A second clinical trial was performed on 476 suspected patient sputum samples at the Pasture Institute of Iran, Department of Mycobacteriology, Tehran, Iran. All patient samples were suspected TB cases and were tested for TB using culture, PCR, AFB stain, and the TB Rapid Test. The clinical status of only 9 cases was known (7 patients had started TB treatment in the week before the patient samples were collected and 2 patients were HIV positive). Among the 7 patients under treatment, 5 patients were culture negative for TB but were positive by direct microscopic examination and a PCR diagnostic test. One patient was AFB stain negative but positive culture methods, a PCR diagnostic test, and the TB Rapid Test. One patient was positive for all four tests. Both HIV positive patients were culture negative but positive for the other three tests. Several other patients were tested repeatedly and, among the positive cases, 11 samples gave false negative culture results.

Diagnosis was based on culture results and clinical status. The results of this clinical trial are shown below in Table 2.

TABLE 2
The Results of the Clinical Trial Performed to Evaluate the TB
Diagnosis of 476 Suspected Tuberculosis Patients

| | True | False | True | False | | | | | |
|---|---|---|---|---|---|---|---|---|---|
| Diagnostic test | pos. | pos. | neg. | neg. | Sensitivity | Specificity | PPV* | NPV* | Efficiency |
| DIAGNOSIS | 151 | 0 | 325 | 0 | — | — | — | — | — |
| Culture | 133 | 0 | 325 | 18 | 88.1 | 100.0 | 100.0 | 94.8 | 96.2 |
| AFB stain | 130 | 2 | 323 | 21 | 86.1 | 99.4 | 98.5 | 93.9 | 95.2 |
| TB rapid test | 147 | 23 | 302 | 4 | 97.4 | 92.9 | 86.5 | 98.7 | 94.3 |

*PPV = Positive Predictive Value, NPV = Negative Predictive Value

Bayes parameters for the culture, the AFB stain, and the TB Rapid Test are given in Table 2. AFB detection by microscopy showed a low sensitivity (86.1%), a good positive predictive value (PPV), and a negative predictive value (NPV) below 95%. The TB Rapid Test had good sensitivity (97.4%) but its specificity was below 95%. Its PPV was thus below 90%, although it had the highest NPV (98.7%) of the three tests. Culture results indicate that the sensitivity of this technique, although higher than that of microscopy, is still below 90%. Its specificity is 100%, resulting in 100% PPV and a NPV close to 95%. The efficiency of the tree methods are around 95%. As the contamination rate in our lab averages 4.8%, the true efficiency of culture can be calculated as 92% for this study.

Clinical trial 3: A third clinical trial was performed on nine patient sputa from Maseeh Daneshvari Hospital. The clinical trial 3 performed the TB Rapid Test using the Ab-MDR preparation (MDR TB Rapid Test) that binds specifically to MDR strains of TB on sputa samples of nine patients with a suspected clinical history of MDR tuberculosis. The results of this MDR TB Rapid Test on controls and known MDR strains of TB are shown in Table 3 below.

TABLE 3
Results of MDR TB Rapid Test on Control Samples

| Sample | H₂0 | PBS | BCG | Sp− | Sp− | K4 RR | RIF 80 |
|---|---|---|---|---|---|---|---|
| 100 μl | C− | C− | C− | C− | C− | C+ | C+ |
| Result | − | − | − | − | − | + | ± |
| 200 μl | C− | C− | C− | C− | C− | C+ | C+ |
| Result | − | − | − | − | − | + | + |

The negative controls were water, phosphate buffered saline, M. bovis cells, and two sputum samples known to be negative for MDR TB. The two positive controls were samples spiked with known MDR strains of M. tuberculosis. Both the K4 RR strain and the RIF 80 strain are resistant to rifampicin and isoniazid. The MDR TB Rapid Test gave negative results to all of the negative controls and it gave positive results to the two samples spiked with known MDR TB cells at about 2×10⁴ cells/ml.

The results for the nine patient sputa are given in Table 4, but these results are hard to verify since an absolute diagnosis of the MDR status of the patient's TB was not available.

Discussion

The best diagnostic tool available for TB detection is generally accepted to be culture methods. Indeed, most authors report a good sensitivity (90-100%) for this method in pulmonary cases (12), but the total number of positive TB cases that are culture negative in the U.S.A reached 22% in 2005 (17) and certain studies report sensitivities of 50% or lower (7,9,18) in special patient groups. While culture methods remain the gold standard for TB, the length of time required before obtaining results and the equipment required to perform this technique makes it an inadequate diagnostic tool for use in most TB endemic areas. Thus the AFB microscopy is generally advocated in its place.

The lower positive predictive value (PPV) of the TB Rapid Test implies that some non-TB infected patients will be thought to be TB positive while the much higher negative predictive value (NPV) of the TB Rapid Test implies that the subjects found to be negative are almost certainly exempt of TB.

In standard practice, confirmatory tests—such as AFB microscopy, culture test, PCR or X-rays will be requested by the physicians when receiving positive TB results for subjects without clinical TB signs. This is far less dangerous than allowing a true positive case that was falsely diagnosed as negative on the basis of its AFB smear results to reintegrate into the general population and become a danger to others

The calculated sensitivity and specificity of AFB microscopy and culture during this trial are similar to what is reported in the literature for adult pulmonary tuberculosis. There is no statistical difference (p=0.05) between the efficiency of the AFB microscopy, the Culture method or the TB Rapid Test in the studies described herein. The TB Rapid Test made it to be the most sensitive yet the least specific of all three methods. Yet the high negative predictive value of the TB Rapid Test makes it the best screening test of all the available TB diagnostic assays.

It should be appreciated by those skilled in the art that the conception and the specific embodiment disclosed might be readily utilized as a basis for modifying or redesigning the medical examination device for carrying out the same purposes as the invention. It should be realized by those skilled in the art that such equivalent constructions do not depart from the spirit and scope of the invention as set forth in the appended claims.

### REFERENCES

All patents and publications mentioned in this specification are indicative of the level of skill of those of knowledge in the art to which the invention pertains. All patents and publications referred to in this application are incorporated herein by reference to the same extent as if each was specifically indicated as being incorporated by reference and to the extent that they provide materials and methods not specifically shown.
1. Global Tuberculosis Programme. 1999. Laboratory Services In Tuberculosis Control. Part I: Organization and Management. WHOffB198.258. 63 pg. World Health Organization (Ed). Geneva, Switzerland.
2. Global Tuberculosis Programme. 1999. Laboratory Services In Tuberculosis Control. Part II Microscopy WHO/TB/98.258. 61 pg. World Health Organization (Ed). Geneva, Switzerland.
3. Global Tuberculosis Programme. 1999. Laboratory Services In Tuberculosis Control. Part III: Culture WHO/TB/98.258. 95 pg. World Health Organization (Ed). Geneva, Switzerland.
4. American Thoracic Society. 2000. Diagnostic Standards and Classification of Tuberculosis in Adults and Children. Am. J. Respir. Crit. Care Med. 161: 1376-1395.
5. International Union Against Tuberculosis and Lung Disease (Laszlo A, ed.). 2000. Technical guide. Sputum examination for tuberculosis by direct microscopy in low income countries (Fifth edition), International Union Against Tuberculosis and Lung Disease, Paris, France.
6. Rieder H L, Chonde T M, Myking H, et al. 1998. The public health service national reference laboratory and the national laboratory network. Minimum requirements, role and operation in a low-income country. International Union Against Tuberculosis and Lung Disease, Paris, France.
7. Chiang H, Suo J, Bai K-J, Lin T-P. 1997. Serodiagnosis of tuberculosis. A study comparing three specific mycobacterial antigens. Am. J. Respir. Crit. Care Med. 156:906-911.
8. Zou Y L, Mang J D, Chen M J-I, Shi Q G. 1994. Serological analysis of pulmonary and extrapulmonary tuberculosis with enzyme-linked immunosorbent assays for A60 Immunoglobulins. Clin. Infect. Dis. 19: 1084-1091.
9. Swarminathan S, Umadcvi P. 1999. Sero diagnosis of tuberculosis in children using two ELISA kits. Indian J. Pediatr. 6:837-842.
10. Kim Te, Blackman R S, Heatwole K M el al. 1984. Acid-fast bacilli in sputum smears of patients with pulmonary tuberculosis: prevalence and significance of negative smears pretreatment and positive smears post-treatment. Am. Rev. Respi. Dis. 129:264-268.
11. Delacourt C, Gobin J, Gaillard J L, de Blic J. [993. Value of ELISA using antigen A60 for the diagnosis of tuberculosis in children. Chest 104:393-398.
12. Lu K-T, Yu C-J, Yang P-C. 1996. Tuberculosis antigen A60 serodiagnosis of tuberculosis infection: application in extrapulmonary and smear-negative pulmonary tuberculosis. Respirology 1:145-151.
13. Pandiani Maes R. 1993. Significato delle misurazioni sierologiche ottenute nelle infezioni microbatteriche. Biologia Clinica. 23(4): 15-27.
14. Gupta S. Kumari S. Banwalikar I N, Gupta S K. 1995. Diagnostic utility of the estimation of mycobacterial Antigen 60 specific immunoglobulins IgM, IgA and IgG in the sera of cases of adult human tuberculosis. Tubercle Lung Dis. 76:418.424.
15. Hobby G L, Holman A P. Iseman M O, Jones J. 1973. Enumeration of tubercle bacilli in sputum of patients with pulmonary tuberculosis. Antimicrob. Agents Chemother. 4:94-104.
16. Yeager H I, Lacy J, Smith L R, LeMaistre C. 1967. Quantitative studies on mycobacterial populations in sputum and saliva. Am. Rev. Respir. Dis. 95:998-1004.
17. Centers for Disease Control and Prevention. National Center for HIV, STD and TB Prevention. Tuberculosis Surveillance Reports. Reported tuberculosis in the United States, 2005. Table 8: tuberculosis cases and percentages by case verification criterion and site of disease: United States 1993-2005. U.S. Department of Health and Human Services, Public Health Service, Atlanta, Ga., USA, 2006.
18. Burman W J, Stone B L, Reyes R R, Wilson M L, Yang Z, El-Hajj H, Bates J H, Cave M D. The incidence of false-positive cultures for Mycobacterium tuberculosis. Am J Respir Crit Care Med 1997; 155(1); 321-326.

What is claimed is:

1. An immunoassay for screening patient samples for Mycobacterium tuberculosis, the immunoassay having an antiserum against a plurality of cell wall antigens isolated from a multitude of Mycobacterium tuberculosis strains.

2. The immunoassay of claim 1, wherein the cell wall antigens are isolated from a cell bank comprising Mycobacterium tuberculosis cells from at least 100 strains.

3. The immunoassay of claim 1, wherein the cell wall antigens are isolated from a cell bank comprising Mycobacterium tuberculosis cells from at least 100 strains of multidrug resistant Mycobacterium tuberculosis.

4. The immunoassay of claim 1, wherein the cell wall antigens are isolated from a washed Mycobacterium tuberculosis cell pellet that is alternately placed in liquid nitrogen and boiling water.

5. The immunoassay of claim 4, wherein the cell pellet is alternately placed in liquid nitrogen and boiling water ten times to fracture a multitude of Mycobacterium tuberculosis cell walls in the cell pellet.

6. The immunoassay of claim 5, wherein the cell wall antigens are solubilized from the fractured cell walls of Mycobacterium tuberculosis suspended in a buffered detergent by sonication.

7. The immunoassay of claim 1, wherein the isolated cell wall antigens include one or more proteins having a molecular weight of 18.5 KD, 44 KD, 66.2 KD, 76 KD, 116 KD or 135 KD.

8. The immunoassay of claim 1, wherein the immunoassay has a negative predictive value for the presence of Mycobacterium tuberculosis cell in a patient sample of 95% or more.

9. The immunoassay of claim 6, wherein the cell wall antigens include a variety of proteins selected from a group of proteins having a molecular weight of 18.5 KD, 44 KD, 66.2 KD, 76 KD, 116 KD, 135 KD, and a mixture thereof.

10. The immunoassay of claim 9, wherein one or more unwanted antibodies to a multitude of antibiotic sensitive Mycobacterium tuberculosis cells have been removed from the antiserum.

11. The immunoassay of claim 9, wherein one or more unwanted antibodies to one or more patient sputa samples diagnosed as negative for strains of Mycobacterium tuberculosis by culture have been removed from the antiserum.

12. An immunoassay for screening patient samples for a multidrug resistant strain of Mycobacterium tuberculosis, the immunoassay having an antiserum against a plurality of cell wall antigens isolated from a multitude of multidrug resistant Mycobacterium tuberculosis strains.

13. The immunoassay of claim 12, further comprising a negative control comprising a patient sputa sample diagnosed as negative for multidrug resistant strains of Mycobacterium tuberculosis by culture.

14. The immunoassay of claim 12, further comprising a positive control comprising a plurality of Mycobacterium tuberculosis cells from a cell bank comprising at least 100 strains of multidrug resistant Mycobacterium tuberculosis cells.

15. The immunoassay of claim 12, wherein one or more unwanted antibodies to a multitude of antibiotic sensitive Mycobacterium tuberculosis cells have been removed from the antiserum.

16. The immunoassay of claim 15, wherein one or more unwanted antibodies to one or more patient sputa samples diagnosed as negative for strains of Mycobacterium tuberculosis by culture have been removed from the antiserum.

17. The immunoassay of claim 12, wherein the cell wall antigens are isolated from a washed cell pellet of multidrug resistant Mycobacterium tuberculosis cells by alternately placing the cell pellet in liquid nitrogen and boiling water several times to fracture a multitude of Mycobacterium tuberculosis cell walls in the cell pellet, solubilizing the antigens from the fractured cell walls of Mycobacterium tuberculosis suspended in a buffered detergent by sonication, and separating one or more proteins having a molecular weight of 18.5 KD, 44 KD, 66.2 KD, 76 KD, 116 KD or 135 KD by gel filtration.

18. The immunoassay of claim 12, further comprising a positive control, a negative control, and a reporter conjugate.

19. The immunoassay of claim 12, wherein one or more unwanted antibodies to one or more patient sputa samples diagnosed as negative for strains of Mycobacterium tuberculosis by culture have been removed from the antiserum and wherein one or more unwanted antibodies to a multitude of antibiotic sensitive Mycobacterium tuberculosis cells have been removed from the antiserum.

20. The immunoassay of claim 12, wherein the multitude of multidrug resistant Mycobacterium tuberculosis strains were grown on media containing rifampicin and isoniazid.
